(19) United States
(12) Patent Application Publication
(10) Pub. No.: US 2007/0224676 A1
(43) Pub. Date: Sep. 27, 2007

(54) EXPANDABLE CULTURE ROLLER BOTTLE

(75) Inventor: Tariq A. Haq, Westwood, MA (US)

Correspondence Address: DAVID W. HIGHET VP & CHIEF IP COUNSEL;BECTON, DICKINSON AND COMPANY, (Hoffman & Baron), 1 BECTON DRIVE, MC 110, FRANKLIN LAKES, NJ, 07417-1880, US

(73) Assignee: BECTON, DICKINSON AND COMPANY, Franklin Lakes, NJ (US)

(21) Appl. No.: 11/688,491

(22) Filed: Mar. 20, 2007

Related U.S. Application Data
(60) Provisional application No. 60/743,608, filed on Mar. 21, 2006.
(60) Provisional application No. 60/864,067, filed on Nov. 2, 2006.

(51) Int. Cl. C12M 1/24

(52) U.S. Cl. 435/298.2; 435/304.1

(57) ABSTRACT

The present invention provides a roller bottle for cell growth culturing, the roller bottle including a bottom wall; a top wall having an opening formed therethrough; and, a side wall extending between the top and bottom walls, a plurality of corrugations being defined in the side wall. Further, a distance is defined between the top and bottom walls, and the corrugations are selectively deformable to alter the distance. Advantageously, with the present invention, a single roller bottle may be provided with its length being adjustable in response to cell culture size and density.

### CROSS-REFERENCES TO RELATED APPLICATIONS

This application claims priority of U.S. Provisional Patent Application No. 60/743,608, filed Mar. 1, 2006. and of U.S. Provisional Patent Application No. 60/864,067, filed Nov. 2, 2006, the entireties of which are incorporated by reference herein.

### FIELD OF INVENTION

This invention relates to a container for cell culture production, and, more particularly, to a roller bottle having an extendible and/or contractible corrugated surface for altering the length of the roller bottle in response to cell culture size and density and compactability For ease of shipping.

### DESCRIPTION OF RELATED ART

One type of container commonly used in a laboratory for culturing cells is known as a “roller bottle”. Roller bottles are generally cylindrical and are adapted to rotate about their axes. The internal surfaces of such roller bottles provide active surfaces for cells. A liquid growth medium is introduced into the roller bottle to promote cell growth. The rotating movement of the bottle keeps the internal surfaces wetted with the liquid medium, thereby encouraging the growth of cells. Once grown, the cells may be harvested and used in different applications.

Cell growth within the roller bottle depends on different factors, including maintainable cell density. Typically, cell cultures are grown by initially seeding the roller bottle with the desired cell and providing growth media. Large cell cultures are achieved through iterative growing stages. These growing stages may be accomplished with increasingly larger cell cultures being introduced into increasingly larger containers so that relatively high cell densities may be maintained throughout the growth process.

### SUMMARY OF THE INVENTION

The present invention provides a roller bottle for cell growth culturing, the roller bottle including a bottom wall; a top wall having an opening formed therethrough; and, a side wall extending between the top and bottom walls, a plurality of corrugations being defined in the side wall. Further, a distance is defined between the top and bottom walls, and the corrugations are selectively deformable to alter the distance. Advantageously, with the present invention, a single roller bottle may be provided with its length being adjustable in response to cell culture size and density.

These and other features of the invention will be better understood through a study of the following detailed description and accompanying drawings.

### BRIEF DESCRIPTION OF THE DRAWINGS

FIG. 1 is a plan view of a roller bottle formed in accordance with the subject invention;

FIG. 2 is a partial cross-sectional view taken along line 2-2 of FIG. 1;

FIGS. 3(a) and 3(b) depict schematically the contraction and expansion of a roller bottle formed in accordance with the subject invention; and,

FIGS. 4(a) and 4(b) depict schematically a roller bottle formed in accordance with the subject invention in use.

### DETAILED DESCRIPTION OF THE INVENTION

As shown in the FIGS., a roller bottle 10 is provided having a top wall 12, a side wall 14, and a bottom wall 16. Preferably, the roller bottle 10 is unitarily formed. The top wall 12, the side wall 14, and the bottom wall 16 collectively define an interior volume 18 in which cell cultures can be grown.

A plurality of corrugations 20 are defined in the side wall 14 which are selectively deformable to alter the distance between the top wall 12 and the bottom wall 16. As best shown in FIG. 2, each corrugation 20 is defined by two corrugation walls 22 joined by an outer joint 24. Adjacent corrugations 20 are joined by an inner joint 26. A partial corrugation 28 may be located at one or both ends of the corrugations 20 which is defined by one, or a portion thereof, of the corrugation walls 22.

The corrugations 20 collectively have an accordion-type structure which is selectively deformable. The corrugations 20 may be selectively extended to increase the distance between the top wall 12 and the bottom wall 16. In addition, the corrugations 20 may be selectively contracted to decrease the distance between the top wall 12 and the bottom wall 16. FIG. 2 shows in dashed line a contracted state of the corrugations 20 as compared to the expanded state of the corrugations 20 shown in solid line.

The corrugation walls 22 may be formed with various configurations, including being generally flat and/or arcuate. In addition, the inner and outer joints 24 and 26 may be formed with various configurations, including being rounded, flat, corner-shaped, and so forth. The corrugation walls 22 subtend an interior angle α which is defined on the interior of the side wall 14 within the interior volume 18. Across the range of deformation of the corrugations 20, the interior angle α may be in the range of approximately 0 degrees, when compressed, to approximately 180 degrees, when fully expanded. With the corrugations 20 being extended, the interior angle α will increase; whereas, with the corrugations 20 being contracted, the interior angle α will decrease.

It is preferred that the corrugations 20 maintain their shape when not being deformed. To facilitate deformation of the corrugations and maintenance of their shape once deformed, the side wall 14 may be defined with different configurations depending on the material being used to form it. For example, the side wall 14 may have varying thicknesses, such as being thinner at the inner and outer joints 24 and 26 as opposed to the corrugation walls 22, to facilitate deformation. Alternatively, the side wall 14 may have a continuous thickness throughout. It is preferred that the thickness of the side wall 14 be greater than 0.5 mm, more preferably greater than 1 mm, to maintain clarity and rigidity of the corrugation walls 22 and resilience at the inner and outer joints 24 and 26.

It is preferred that the corrugations 20 extend circumferentially around the roller bottle 10 continuously and without interruptions. The roller bottle 10 may experience pressure build-up in the interior volume 18 during use (e.g. gas pressure build-up) which may stress the roller bottle 10 resulting in a possible bulge or deformation in the roller bottle 10. The continuous and uninterrupted configuration of the corrugations 20 will provide hoop strength to resist such bulging or deformation. In addition, it is preferred that the outer joints 24 be generally straight and be generally perpendicular to a longitudinal axis 30 of the roller bottle 10. It is further preferred that the inner joints 26 be parallel to the outer joints 24. With the preferred configuration of the outer joints 24, the inner joints 26 also will be generally straight and be generally perpendicular to the longitudinal axis 30 of the roller bottle 10.

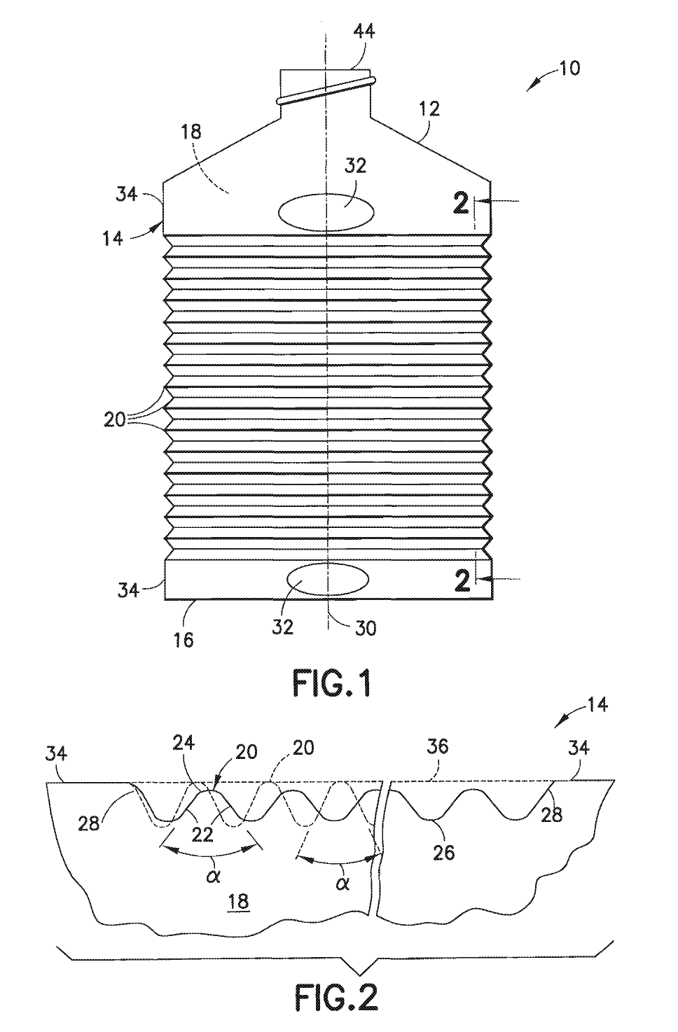

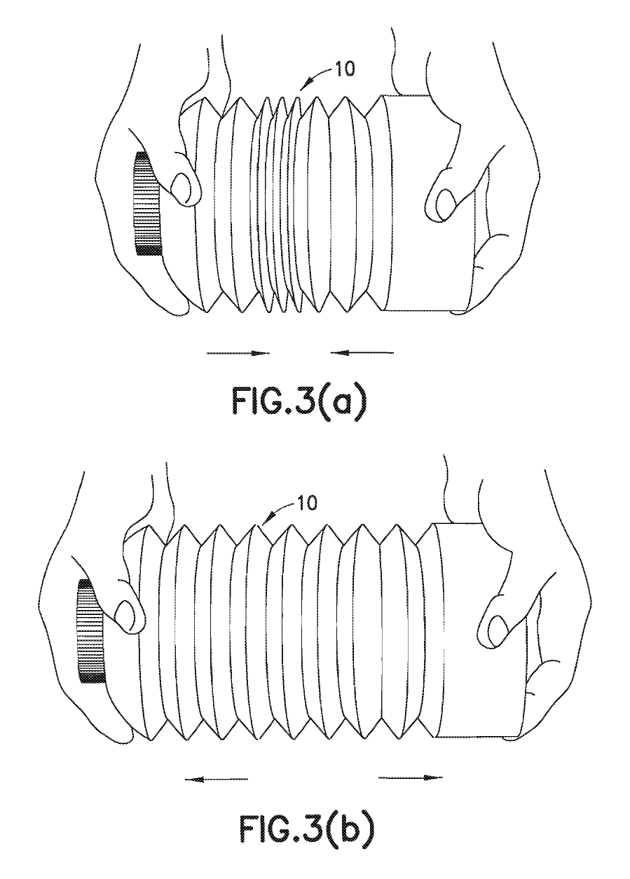

FIGS. 3(a) and 3(b) depict how the roller bottle 10 may be contracted or expanded. To achieve either deformation, the roller bottle 10 may be grabbed at the top wall 12 and the bottom wall 16 by the user. To achieve contraction, the top wall 12 and the bottom wall 16 are pressed together along the longitudinal axis 30 of the roller bottle 10 (FIG. 3(a)). Conversely, to extend the length of the roller bottle 10, the top wall 12 and the bottom wall 16 are pulled apart along the longitudinal axis 30 of the roller bottle 10 (FIG. 3(b)). To facilitate handling of the roller bottle 10 during deformation, one or more indentations 32 (FIG. 1) may be provided in the side wall 14 in proximity to the top wall 12 and/or the bottom wall 16. The indentations 32 may be sized to accommodate one or more of the user's fingers, particularly the thumbs.

The roller bottle 10 must be able to be rolled on its side during cell culture formation. With expansion and contraction of the corrugations 20, the location of the outer joints 24 may vary radially relative to the center of the roller bottle 10. It is thus preferred that flat portions 34 be provided which are defined between the corrugations 20 and the top and bottom walls 12 and 16, respectively. The flat portions 34 may collectively define a stable resting surface 36 for the roller bottle 10 on its side, regardless of the deformed state of the corrugations 20. To ensure maximum stability of the flat portions 34, it is preferred that the outer joints 24 be defined to be coplanar with, or radially inwardly of, the resting surface 36 in the most contracted state of the corrugations 20. With extension of the corrugations 20, the outer joints 24 will be contracted radially inwardly, without disrupting the the resting surface 36. Alternatively, the outer joints 24 may collectively define the resting surface 36, regardless of the state of deformation of the corrugations 20. Thus, the corrugations 20 may be formed to extend beyond the flat portions 34. It is preferred that the outer joints 24 be generally coplanar to define the resting surface 36.

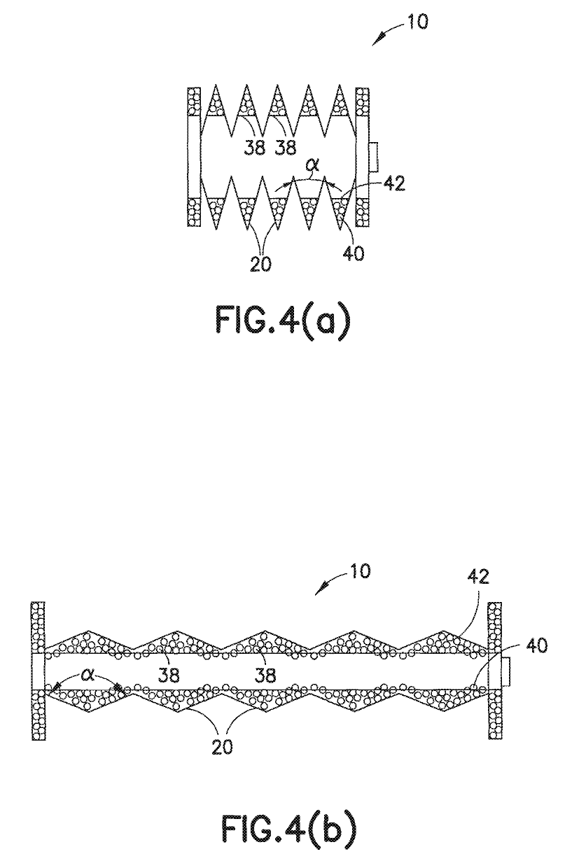

With reference to FIGS. 4(a) and 4(b), an exemplary use of the roller bottle 10 is shown. The roller bottle 10, as shown in FIG. 4(a), is initially provided in a contracted state. As such, the corrugations 20 provide relatively narrow reservoirs 38 for cells 40 and growth media 42, with the interior angle α being relatively low. Once prepared, the roller bottle 10 is rolled on its side supported by the resting surface 36 to facilitate cell growth. When sufficient cell growth has been achieved, the roller bottle 10 may be expanded, with the corrugations 20 becoming wider, the reservoirs 38 becoming shallower, and the interior angle α becoming greater. In this manner, with a relatively small number of the cells 40 being utilized in the initial state of the roller bottle 10 shown in FIG. 4(a), the cells 40 may be located relatively close to the corrugation walls 22, with relatively high cell density being achievable. With sufficient cell growth, cell density can be maintained with more of the cells 40 and/or the media 42 being added in the expanded state of the roller bottle 10 shown in FIG. 4(b).

To facilitate removal of cells, the top wall 12 is provided with an opening 44 (FIG. 1). The opening 44 may be relatively large to allow for easy removal of the cells 40 and scraping of the interior volume 18. The opening 44 may have formed thereabout threads or other connecting configurations to permit attachment of a cap to prevent unwanted leakage from the roller bottle 10 during use.

The roller bottle 10 may be formed of various materials, particularly those conducive to cell growth. A variety of thermoplastic materials may be utilized including, but not limited to, polystyrene, polypropylene, polyethylene terephthalate, polyvinyl chloride, and combinations thereof. As will be recognized by those skilled in the art, other polyolefins may also be utilized. The roller bottle 10 may also be modified to have different surface properties, such as with plasma treatment or coating with chemical or biological agents.

The roller bottle 10 may be formed by blow molding. It is preferred that the roller bottle 10 be formed in an expanded state. The roller bottle 10 may be formed in different sizes and shapes to provide the interior volume 18 in different capacities (e.g., 2.25 liters).

An additional advantage of the roller bottle 10 is that the roller bottle 10 may be stored and transported in its contracted state. Accordingly, higher density packing of the roller bottles 10 during transportation and storage may be achieved as compared to similar capacity roller bottles of the prior art. Furthermore, roller racks used to roll the roller bottles 10 may accommodate greater numbers of the roller bottles 10 in their respective partially or fully contracted states, as compared to prior art roller bottles.

What is claimed is:

1. A roller bottle for cell growth culturing, said roller bottle comprising:
a bottom wall;
a top wall having an opening formed therethrough; and,
a side wall extending between said top and bottom walls, a plurality of corrugations being defined in said side wall, wherein a distance is defined between said top and bottom walls, said corrugations being selectively deformable to alter said distance.

2. A roller bottle as in claim 1, wherein said corrugations are selectively extendible to increase said distance.

3. A roller bottle as in claim 1, wherein said corrugations are selectively contractible to decrease said distance.

4. A roller bottle as in claim 2, wherein said corrugations are selectively contractible to decrease said distance.

5. A roller bottle as in claim 1, wherein said corrugations maintain their shape when not being deformed.

6. A roller bottle as in claim 1, wherein said side wall is formed with a constant thickness.

7. A roller bottle as in claim 1, wherein said side wall is formed with varying thicknesses.

8. A roller bottle as in claim 1, wherein at least one of said corrugations is at least partially defined by two corrugation walls.

9. A roller bottle as in claim 8, wherein said corrugation walls are generally flat.

10. A roller bottle as in claim 8, wherein said corrugation walls are arcuate.

11. A roller bottle as in claim 8, wherein said corrugation walls are joined by a joint, said joint being generally perpendicular to a longitudinal axis of the roller bottle.

12. A roller bottle as in claim 8, wherein said corrugation walls subtend an interior angle defined internally of said side wall, said interior angle being in the range of approximately 0 degrees to approximately 180 degrees.

13. A roller bottle as in claim 1, wherein the roller bottle is formed from a material selected from the group consisting of polystyrene, polypropylene, polyethylene terephthalate, polyvinyl chloride, and combinations thereof.

14. A roller bottle as in claim 1, wherein the roller bottle is formed of a thermoplastic material.

15. A roller bottle as in claim 1, wherein at least one corrugation extends circumferentially about said side wall continuously and without interruptions.

16. A roller bottle as in claim 1, further comprising at least one indentation formed in said side wall in proximity to said bottom wall.

17. A roller bottle as in claim 16, further comprising at least one indentation formed in said side wall in proximity to said top wall.

18. A roller bottle as in claim 17, wherein said corrugations are defined between said indentations.

19. A roller bottle for cell growth culturing, said roller bottle comprising:
a bottom wall;
a top wall having an opening formed therethrough; and,
a side wall extending between said top and bottom walls, a plurality of corrugations being defined in said side wall, at least one corrugation extending circumferentially about said side wall continuously and without interruptions, said corrugation being at least partially defined by two corrugation walls joined by a joint, said joint being generally perpendicular to a longitudinal axis of the roller bottle, wherein a distance is defined between said top and bottom walls, said corrugations being selectively deformable to alter said distance.
